(19) United States
(12) Patent Application Publication
(10) Pub. No.: US 2007/0073252 A1
(43) Pub. Date: Mar. 29, 2007

(54) Male urinary incontinence undergarment

(76) Inventor: Lynn Forgrave, Campbellford (CA)

Correspondence Address: SEAN KAUFHOLD, P. O. BOX 89626, SIOUX FALLS, SD, 57109, US

(21) Appl. No.: 11/234,800

(22) Filed: Sep. 26, 2005

(51) Int. Cl. A61F 5/44

(52) U.S. Cl. 604/349; 604/347

(57) ABSTRACT

A male urinary incontinence undergarment includes a pair of briefs that has a front side and a rear side. A water impermeable container is attached to the front side. The front side has an opening therein that extends into the container. The pair of briefs may be worn and a penis extended through the opening.

### BACKGROUND OF THE INVENTION

1. Field of the Invention

The present invention relates to incontinence undergarment devices and more particularly pertains to a new incontinence undergarment device that includes a section for receiving a penis and for catching and retaining any urine lost due to male incontinence.

2. Description of the Prior Art

The use of incontinence undergarment devices is known in the prior art. U.S. Pat. No. 5,009,649 describes a condom device that is positionable within an undergarment and which may be positioned over a penis to receive any urine lost due to male incontinence. Another type of incontinence undergarment device is U.S. Pat. No. 4,588,397 having an external catheter than is positionable over a penis for receiving and containing urine. Still yet another such device is found in U.S. Pat. No. 6,419,665.

While these devices fulfill their respective, particular objectives and requirements, the need remains for a device that may be worn as a conventional undergarment but which includes an attached, sealed container into which a penis may be inserted for retaining any lost urine due to male incontinence. Further, the device should preferably include a a sheath through which the penis may be extended so that a seal is formed around the penis.

### SUMMARY OF THE INVENTION

The present invention meets the needs presented above by comprising a pair of briefs that has a front side and a rear side. A container is attached to the front side and comprises a water impermeable material. The front side has an opening therein that extends into the container. The pair of briefs may be worn and a penis extended through the opening.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

The objects of the invention, along with the various features of novelty which characterize the invention, are pointed out with particularity in the claims annexed to and forming a part of this disclosure.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a front perspective view of a male urinary incontinence undergarment according to the present invention.

FIG. 2 is a side view of the present invention.

FIG. 3 is a front perspective view of the present invention.

FIG. 4 is a perspective cross-sectional view of the present invention.

FIG. 5 is a cross-sectional view taken along line 5-5 of FIG. 1 of the present invention.

### DESCRIPTION OF THE PREFERRED EMBODIMENT

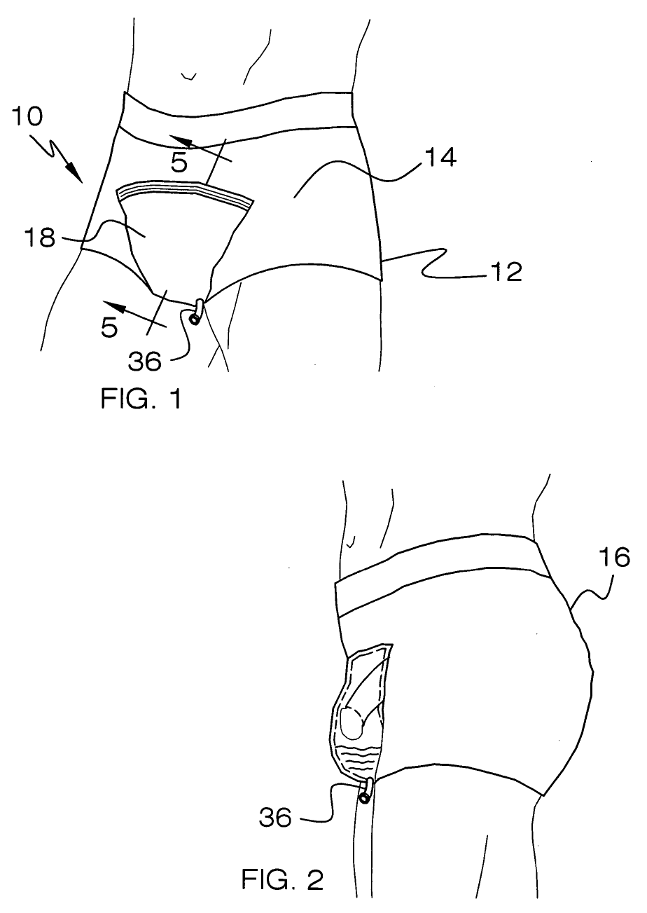

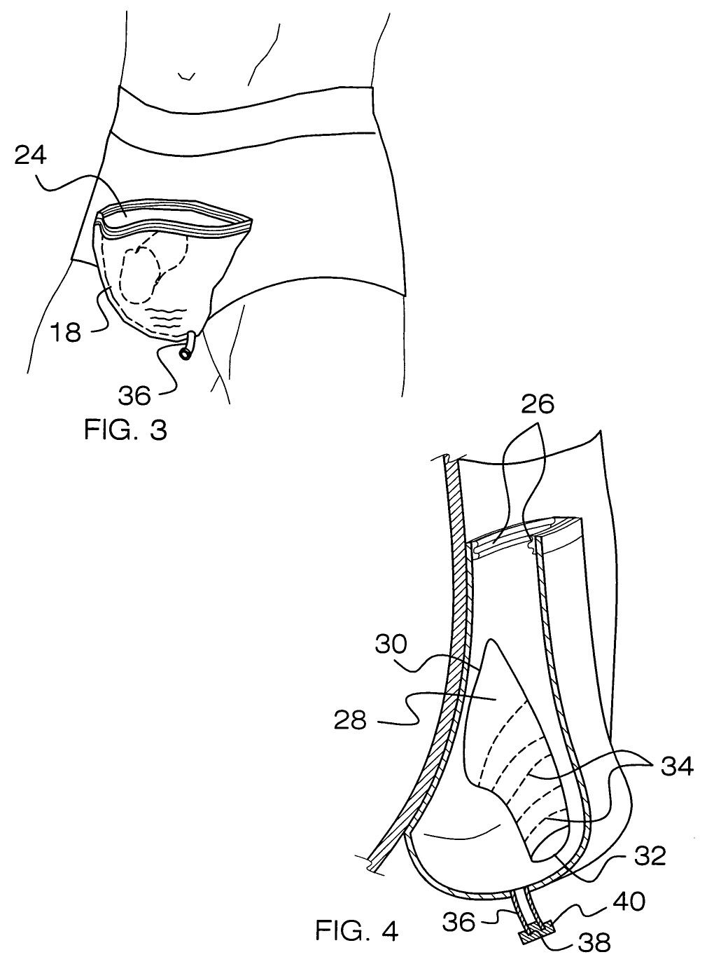

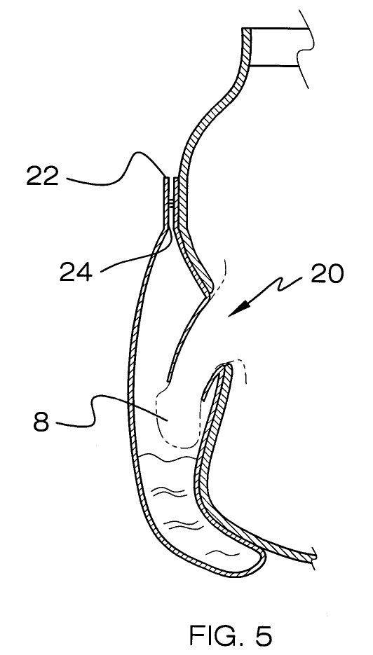

With reference now to the drawings, and in particular to FIGS. 1 through 5 thereof, a new incontinence undergarment device embodying the principles and concepts of the present invention and generally designated by the reference numeral 10 will be described.

As best illustrated in FIGS. 1 through 5, the male urinary incontinence undergarment 10 generally comprises a pair of generally conventional briefs 12 that have a front side 14 and a rear side 16. A container 18 is attached to the front side 14. The container 18 comprises a water impermeable material such as a flexible plastic or an elastomer. The front side 14 has an opening 20 therein. The opening 20 extends into the container 18. An upper edge 22 of the container 18 has an elongated slit 24 therein. A closure 26 is attached to and is coextensive with the slit 24 for selectively sealing the slit 24.

A sleeve 28 has a first end 30 and a second end 32. The first end 30 is attached to the container 18 and extends along a peripheral edge of the opening 20. The sleeve 28 has a plurality of perforation rings 34 therein for selectively reducing a length of the sleeve 28.

A conduit 36 is fluidly coupled to the container 18 and extends away from a bottom portion of the container 18. A cap member 40 is removably positionable over and closes a free end 38 of the conduit 36.

In use, the pair of briefs 12 may be worn and a penis 8 extended through the opening 20. The sleeve 28 may be shortened along a selected one of the perforation rings 34 so that the penis 8 extends outwardly of the second end 32 of the sleeve 28. The sleeve 28 is preferably comprised of a resilient material, such as an elastomer, for forming a seal around the penis 8. Any urine captured in the container 18 may be drained through the conduit 36 or the slit 24, though the slit 24 is primarily useful for accessing the sleeve 28.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A urine receptacle assembly for male incontinence, said assembly comprising:
a pair of briefs having a front side and a rear side;
a container being attached to said front side, said container comprising a water impermeable material, said front side having an opening therein, said opening extending into said container, an upper edge of said container has an elongated slit therein, a closure being attached to and being coextensive with said slit for selectively sealing said slit;
a sleeve having a first end and a second end, said first end being attached to said container and extending along a peripheral edge of said opening, said sleeve being positioned within said container;
a conduit being fluidly coupled to said container, a cap member being removably positionable over and closing a free end of said conduit, said conduit being spaced from said elongated slit; and
wherein said pair of briefs may be worn and a penis extended through said opening.

2-3. (canceled)

4. The assembly according to claim 1, wherein said sleeve has a plurality of perforation rings therein for selectively reducing a length of said sleeve.

5-6. (canceled)

7. A urine receptacle assembly for male incontinence, said assembly comprising:
a pair of briefs having a front side and a rear side;
a container being attached to said front side, said container comprising a water impermeable material, said front side having an opening therein, said opening extending into said container, an upper edge of said container having an elongated slit therein, a closure being attached to and being coextensive with said slit for selectively sealing said slit;
a sleeve having a first end and a second end, said first end being non-removably attached to said container and extending along a peripheral edge of said opening, said sleeve having a plurality of perforation rings therein for selectively reducing a length of said sleeve, said sleeve being positioned within said container;
a conduit being fluidly coupled to said container and spaced from said slit, a cap member being removably positionable over and closing a free end of said conduit; and
wherein said pair of briefs may be worn and a penis extended through said opening.

8. A urine receptacle assembly for male incontinence, said assembly comprising:
a pair of briefs having a front side and a rear side;
a container being attached to said front side, said container comprising a water impermeable material, said front side having an opening therein, said opening extending into said container;
a sleeve having a first end and a second end, said first end being non-removably attached to said container and extending along a peripheral edge of said opening, said sleeve being positioned within said container; and
wherein said pair of briefs may be worn and a penis extended through said opening.
